(19) United States
(12) Patent Application Publication
(10) Pub. No.: US 2022/0240904 A1
(43) Pub. Date: Aug. 4, 2022

(54) ADJUSTABLE TRANSPERINEAL BIOPSY NEEDLE GUIDE

(71) Applicants: Amit Patel, Grosse Pointe, MI (US); Craig Rogers, Plymouth, MI (US); Alexandra Moceri, Troy, MI (US)

(72) Inventors: Amit Patel, Grosse Pointe, MI (US); Craig Rogers, Plymouth, MI (US); Alexandra Moceri, Troy, MI (US)

(21) Appl. No.: 17/649,410

(22) Filed: Jan. 31, 2022

Related U.S. Application Data
(60) Provisional application No. 63/144,323, filed on Feb. 1, 2021.

(51) Int. Cl. A61B 10/02; A61B 17/34

(52) U.S. Cl. CPC A61B 10/0233; A61B 2017/3407; A61B 2017/3413; A61B 17/3403

(57) ABSTRACT

A biopsy needle guide comprises a base, a flexible strap, a bracket, a needle guide bracket, and several locking mechanisms. The base has a first locking mechanism and a concave inner surface that conforms with an ultrasound probe. The flexible strap has a first end coupled to the base and a second free end coupled to the base with the first locking mechanism. The bracket is slidably disposed in a slotted region in the base. The needle guide bracket is configured to receive a biopsy needle guide and the needle guide bracket is slidably disposed in a slotted region in the bracket. Other locking mechanisms permit or prevent movement of the bracket relative to the base as well as movement of the needle guide bracket relative to the bracket.

### CLAIM OF PRIORITY

The present application is a non-provisional of, and claims the benefit of U.S. Provisional Patent Application No. 63/144,323 (Atty. Docket No. 5139.003PV2) filed on Feb. 1, 2021; the entire contents of which is incorporated herein by reference.

### BACKGROUND

A biopsy is a medical procedure where a tissue sample or sample of cells is removed from the body for analysis. The use of a biopsy needle allows penetration of tissue to facilitate retrieval of the sample from a target location. In some cases, a biopsy needle guide is used to help precisely guide insertion of the needle into the target location.

Transrectal ultrasound-guided biopsy of the prostate remains the current mainstay of diagnosis for prostate cancer in the United States, with over one million biopsies performed every year. However, the there is an associated risk of infection (associated risk of infection up to approximately 10%) and an associated risk of sepsis (about 0.6% in the state of Michigan) using this route.

A number of biopsy needle guide inventions are disclosed in the patent literature, including but not limited to US Patent Publication Nos. 2016/0100897; 2015/0366544; and U.S. Pat. Nos. 5,494,039; 7,604,645; 5,976,092; and 5,957,935.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a side view of an example of a biopsy needle guide.

FIG. 2 shows an end view of the biopsy needle guide in FIG. 1.

FIG. 3 shows a side view of a needle guide bracket from the biopsy needle guide in FIG. 1.

FIG. 4 shows an opposite side view of the needle guide bracket in FIG. 3.

FIG. 5 shows an end view of the needle guide bracket in FIGS. 3 and 4.

FIG. 6 shows basic male anatomy.

FIG. 7 shows an example of using a transperineal biopsy needle guide such as the one illustrated in FIG. 1.

FIG. 8 shows another example of a biopsy needle guide.

FIGS. 9A-9D shows still another example of a biopsy needle guide.

FIG. 10A illustrates a perspective view of an example of a biopsy needle guide.

FIG. 10B illustrates another perspective view of the example in FIG. 10A.

FIG. 10C shows and end view of the example in FIGS. 10A-10B.

FIG. 11 shows a perspective view of an example of a bracket from the device shown in FIGS. 10A-10C.

FIG. 12 shows a perspective view of an example of a strap from the device shown in FIGS. 10A-10C.

FIG. 13A shows a perspective view of an example of a needle guide bracket from the device shown in FIGS. 10A-10C.

FIG. 13B shows another perspective view of the needle guide bracket in FIG. 13A.

FIG. 14A shows a perspective view of an example of a base from the device shown in FIGS. 10A-10C.

FIG. 14B shows another perspective view of the base in FIG. 14A.

FIG. 14C shows an end view of the base in FIGS. 14A-14B.

### DETAILED DESCRIPTION

Transrectal ultrasound-guided biopsy of the prostate remains the current mainstay of diagnosis for prostate cancer in the United States, with over one million biopsies performed every year. However, the there is an associated risk of infection (up to approximately 10%) and an associated risk of sepsis (about 0.6% in the state of Michigan) using this access route. This is leading to a gradual shift to performing prostate biopsy via a transperineal route (TPbx) with recent studies suggesting that TPbx is associated with a 0% risk of severe sepsis.

Growing concerns about infection as well as increased antibiotic stewardship also has encouraged the increased use of the transperineal biopsy route. While this route has been traditionally performed in the operating room, newer equipment and procedures allow the transperineal route to be performed in an outpatient setting under local anesthesia.

Several different techniques for TPbx have been reported. Freehand ultrasound guided biopsy is a low-cost procedure that can be performed under local anesthesia. However, it can be difficult to trace the needle due to movement in three dimensions. A grid-based approach allows systematic biopsies to be performed on a stable platform but requires multiple puncture sites of the perineum and requires that patients be sedated and also requires the use of a stepper for setup. A stepper is a fixture that is used to hold the ultrasound probe and also provides a guidance grid for biopsy needle insertion.

Other commercially available devices allow tracking of the needle in parallel to the ultrasound probe, are easy to use and can be performed under local anesthesia and are adaptable to different ultrasound probes. However, these devices may be costly on a per usage basis and may be difficult to move the needle from the posterior to anterior positions. Other reusable perineal prostate guides may allow tracking of the needle in parallel to the ultrasound probe, are easy to use, easy to move the needle from the anterior to posterior positions for biopsy and can be done under local anesthesia and are reusable thereby reducing costs. However, these devices may be useable only with a specific manufacturer's setup, may require a coaxial needle and can be difficult to move closer to the skin once fixed in place.

Therefore, it would be desirable to provide improved biopsy needle guide devices that address at least some of the challenges with existing devices. For example, it may be desirable to provide a device which is reusable, or the device may be single use. It may also be desirable to provide a device that can fix in place a coaxial biopsy needle. It may also be desirable to provide a guide device that allows the biopsy needle to be easily moveable between anterior and posterior positions without requiring removal of the needle every time. It may be desirable to provide a guide device that allows the needle to be easily advanced or retraced relative to the patient's skin, and it may be desirable to provide a device that cooperates with many different types of ultrasound probes. At least some of these challenges will be addressed by the examples disclosed herein.

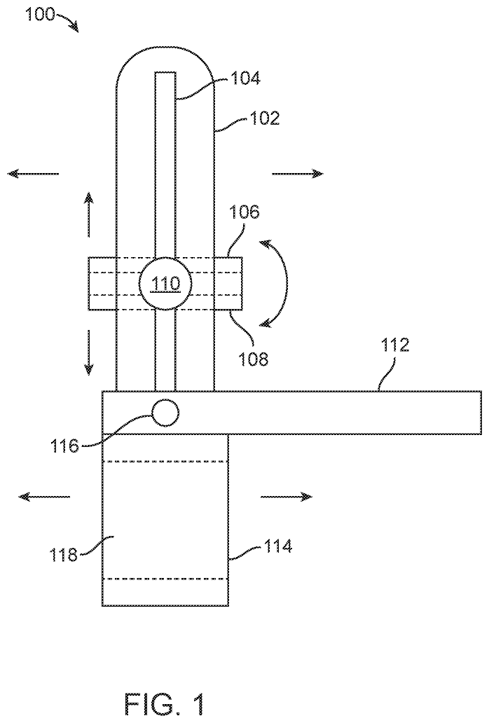

FIG. 1 illustrates a front view of a system 100 in accordance with at least one example of a biopsy needle guide. The biopsy needle guide 100 includes a sliding bracket 102, a needle guide bracket 106, an arm 112, and an ultrasound probe connector 114 (also referred to herein as an ultrasound coupling element). Sliding bracket 102 includes an elongate slot 104, needle guide bracket 106 includes a central channel 108, and ultrasound probe connector 114 also includes a central channel 118. A locking element 110 secures the needle guide bracket 106 to sliding bracket 102. Another locking element 116 secures the ultrasound probe connector 114 and the sliding bracket 102 to the arm 112. Central channel 118 is sized to receive and hold a variety of different sized ultrasound probes (not illustrated).

In FIG. 1, ultrasound probe connector 114 may be coupled to sliding bracket 102, and the assembly of the ultrasound probe connector 114 and the sliding bracket 102 may slide linearly along arm 112 together, or they may be independently movable relative to one another. Locking element 116 may be a set screw, a spring-loaded pin, or any other mechanism for fixing the position of the sliding bracket 102 and the ultrasound probe connector 114 on the arm 112 once the desired position is obtained. Movement of the sliding bracket 102 and ultrasound probe connector 114 along the arm 112 will advance the needle guide bracket 106 closer to the patient's skin (e.g. perineum) or move the needle guide bracket away from the skin (and therefore correspondingly move the biopsy needle closer to or away from the perineum) as desired by the operator. Once in position, locking element 116, here a set screw may be threadably tightened or otherwise locked, thereby locking the position. The ultrasound probe connector 114 and sliding bracket 102 may be orthogonal or otherwise transverse to the longitudinal axis of the arm 112.

Ultrasound probe connector 114 may be a closed ring or an open ring and may be adjustable so that channel 118 is sized to receive and hold a number of different sized ultrasound probes. The adjustment mechanism may be a releasable pawl/teeth mechanism (e.g. a releasable zip tie), or a threaded hose clamp connector, or any other adjustable mechanism known in the art. The ultrasound probe connector 114 may be fixedly attached to the sliding bracket 102, or the two elements may be releasably coupled together and independently movable along arm 112. They may move together along arm 112, or they may move separately as indicated by the horizontal arrows in FIG. 1.

Arm 112 is an elongate arm that supports the ultrasound probe connector 114 and the sliding bracket 102. The arm may have any number of cross-sections such as round, square, rectangular, etc. and its size and shape may be adjusted in order to provide the desired amount of strength to the device while minimizing unwanted weight and material costs. The longitudinal axis of arm 112 in this example is parallel to the longitudinal axis of the ultrasound probe. In other examples the arm 112 may be angled relative to the longitudinal axis of the ultrasound probe and that angle may be any desired angle.

Sliding bracket 102 is an elongate bracket that may be any shape such as rectangular, square, etc. Here, the sliding bracket 102 is an elongate rectangular shaped bracket with a longitudinal axis. An upper end of the sliding bracket has a rounded end to prevent operator injury or inadvertent tears in a physician's or other operator's gloves due to sharp edges. An elongate slot, here a rectangular slot is parallel with the longitudinal axis of the sliding bracket and allows the needle guide bracket 106 to slide up and down along the sliding bracket 102 as indicated by the vertical arrows in FIG. 1. Any of the rectangular slots disclosed in this example or anywhere herein may have ninety degree square corners, or the rectangular slots may have rounded ends or any other shaped ends. Anatomically, sliding the needle guide bracket 106 up or down moves the needle guide anatomically anterior (e.g. toward the perineum or scrotum) and anatomically posterior (e.g. toward the rectum), respectively. Also, in this example, the longitudinal axis of the sliding bracket is orthogonal to the longitudinal axis of the ultrasound probe. In other examples the longitudinal axis of the sliding bracket may be angled relative to the longitudinal axis of the ultrasound probe. The angle may be any desired angle.

Needle guide bracket 106 includes a central channel 108 extending through the needle guide bracket and is sized to receive and hold a biopsy guide needle (not shown) and the biopsy needle is inserted into the biopsy guide needle. In this example or any other example, optionally a sharp inner obturator may be inserted into the biopsy needle guide to help pierce and pass through the tissue and once this is accomplished the inner obturator may be removed and replaced with the biopsy needle. In addition to moving up/down (or anterior/posterior), the needle guide bracket can pivot clockwise or counterclockwise as indicated by the rotational arrows in FIG. 1. Thus, once the needle guide bracket has been disposed in a desired position (e.g. anterior/posterior and rotationally), the locking element 110 may be releasably locked holding the needle guide bracket in position. Here, the locking element may be a set screw, or any other locking mechanism known in the art. If the needle guide bracket position needs to be adjusted, the locking element may be unlocked and then the needle guide bracket position can be re-adjusted and then re-locked. The locking element may also be used to lock the biopsy guide needle into the channel 108, or optionally another locking element such as a set screw may be used to independently lock the biopsy guide needle into the channel of the biopsy needle guide (not shown in FIG. 1). An example of the two locking elements on the needle guide bracket is shown later in this specification. In this example, channel 108 may be colinear (e.g. parallel) with the longitudinal axis of the arm 112 and the longitudinal axis of the ultrasound probe. And thus, since the biopsy guide needle and biopsy needle are inserted into the channel 108, they also are parallel or colinear with the arm 112 and ultrasound probe. The biopsy needle is coaxial with the biopsy guide needle. Even when the needle guide bracket 106 is pivoted, the channel 106 and biopsy guide needle are still colinear with the longitudinal axes of the arm 112 and ultrasound probe in a vertical plane but transverse in the horizontal direction. Therefore, the biopsy guide needle will always be inserted along the same line as the ultrasound probe and this is referred to as being colinear. The biopsy needle is coaxial with the biopsy guide needle allowing a single puncture through the perineum while permitting multiple biopsy tissue samples to be obtained via the single puncture since the biopsy guide needle and biopsy needle may repositioned through the single puncture site.

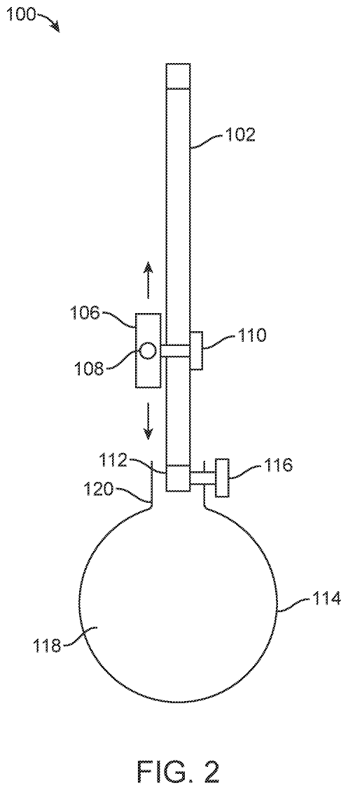

FIG. 2 shows an end view of the biopsy needle guide 100 in FIG. 1. Locking element 110 in this example is a set screw for securing the needle guide bracket 106 to the sliding bracket 102. Channel 108 in the needle guide bracket is sized to receive a biopsy guide needle (not shown) and a biopsy needle is inserted into the biopsy guide needle. As previously disclosed, an optional sharp inner obturator may be positioned in the biopsy guide needle initially to help with tissue penetration and then the obturator is removed and replaced with the biopsy needle. In use, needle guide bracket 106 may be moved up or down along sliding bracket 102 and also pivoted relative to the sliding bracket 102. Once the needle guide bracket 106 is disposed in the desired vertical position and pivoted into a desired rotational position it may be locked into position by tightening locking element 110. Untightening locking element 110 allows the needle guide bracket 102 to move along sliding bracket 102 and pivot. Pivoting allows the biopsy guide needle to enter the patient's skin such as the perineum at different angles and subsequently the biopsy needle will also enter the patient at an angle. The angle may range anywhere from approximately +90 degrees to approximately −90 degrees relative to a line perpendicular to the surface of the perineum. If the needle guide bracket 102 is not pivoted, then the biopsy guide needle and biopsy needle should penetrate the perineum approximately orthogonally to the surface of the perineum. The locking element 110 may optionally also lock a biopsy guide needle into channel 108 or optionally in any example, a second locking element that may take the same form as locking element 110 may be used to lock the biopsy guide needle into the biopsy needle guide bracket independently of locking the biopsy needle guide bracket 106 to the sliding bracket 102. Additional details are disclosed below. As previously discussed, the ultrasound probe connector 114 may be slid along arm 112 to move the needle guide bracket inward or outward relative to the patient's skin. Optional rails 120 are coupled to the ultrasound probe connector 114 to permit the ultrasound probe connector 114 to slide along arm 112. Other connections between the ultrasound probe connector and arm may be used. Once the ultrasound probe connector 114 is disposed in the desired position along arm 112, it may be locked in position by tightening locking element 116 which in this example is a set screw. Releasing the set screw allows the ultrasound probe connector 114 to slide along arm 112. As discussed above, the sliding bracket 102 may be coupled to the ultrasound probe connector 114 and thus the two elements move together along arm 112 when the ultrasound probe connector is translated along the arm 112, and when the ultrasound probe connector is locked, so too is the sliding bracket 102 if coupled together. In other examples, the sliding bracket may not be coupled to the ultrasound probe connector and thus the sliding bracket 102 may be adjusted independently of the ultrasound probe connector 114 along arm 112. In other examples, the ultrasound probe connector 114 may move independently of the sliding bracket 102 along arm 112.

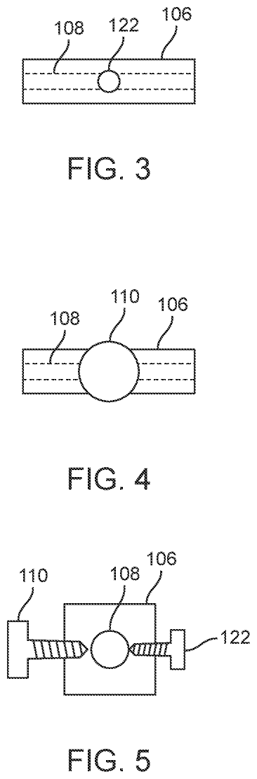

FIG. 3 show a side view of an example of the needle guide bracket 106 which includes a central channel 108 which is sized to receive a biopsy guide needle (not shown). In this example a second locking mechanism is included to secure the biopsy guide needle in place in the central channel 108. Here the locking mechanism 122 may be a set screw, a pin, or any other mechanism for holding the biopsy guide needle. In other examples, the locking mechanism 110 (not seen in this view, but seen in FIG. 4) which holds the biopsy needle bracket 106 to the sliding bracket 102 may also lock the biopsy guide needle into the central channel 108 of the needle guide bracket 106. In other examples, the coaxial biopsy needle guide may snap fit or friction fit into position in the central channel. The coaxial biopsy needle guide may have a tapered outer surface to help create the friction fit and prevent it from sliding out. This may obviate the need for a rubber sleeve that is often placed over the biopsy needle guide to create the friction fit.

FIG. 4 shows a side view of an opposite side of the needle guide bracket 106 in FIG. 3. Here, locking mechanism 110 is on the opposite side of the needle guide bracket 106 and may be used to secure the needle guide bracket 106 to the sliding bracket 102 and/or to secure the biopsy guide needle into channel 108. Here, the locking mechanism is also a set screw.

FIG. 5 shows and end view of the needle guide bracket 106 in FIGS. 3 and 4 above. Here, two locking members 110, 122 are shown. The first locking member 110 is used to secure the guide needle bracket to the sliding bracket 102, and the second locking mechanism 122 secures the biopsy guide needle into channel 108. Both locking mechanisms may be set screws, pins, spring loaded pins, or other mechanisms known in the art.

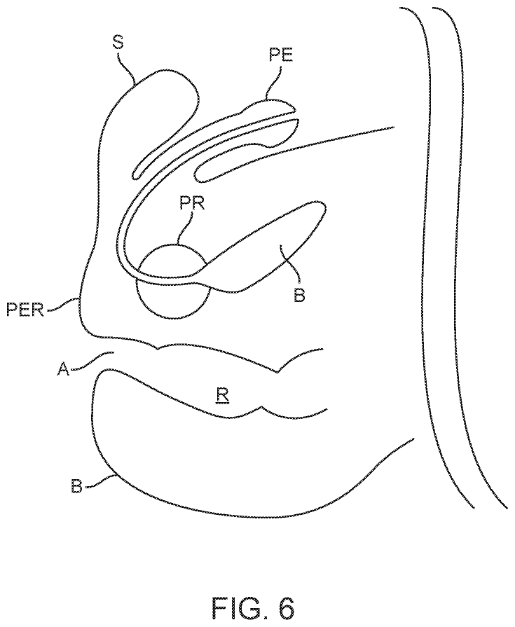

FIG. 6 shows basic male anatomy including bladder B, anus A, rectum R, perineum PER, prostate PR, penis PE, buttocks B, and scrotum S.

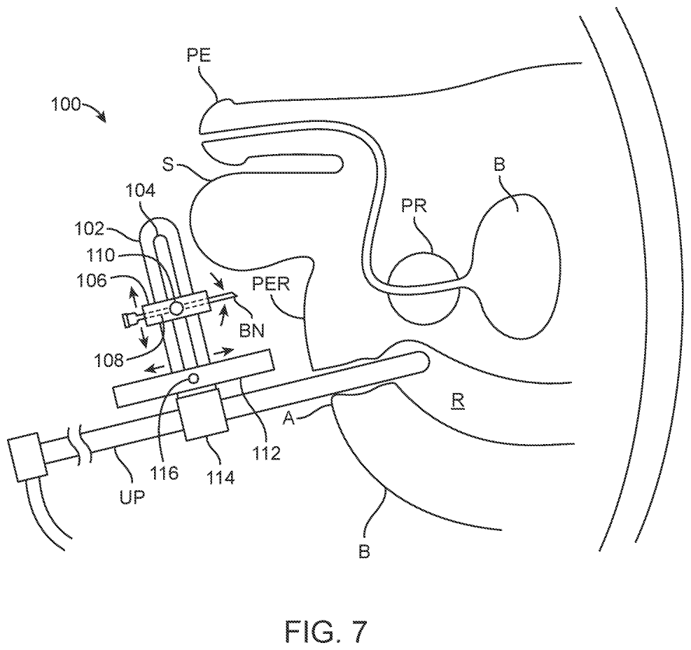

FIG. 7 shows an example of performing a transperineal biopsy needle procedure using a biopsy needle guide such as the example in FIG. 1 or any of the other examples of biopsy needle guides disclosed herein. Here, an ultrasound probe UP is inserted into the patient's anus and advanced through the rectum R. The ultrasound probe allows the operator to guide the biopsy guide needle BN as is it is advanced through the perineum into the prostate or adjacent tissue to ensure that the biopsy sample is obtained at the desired location in the prostate PR as well as avoiding damaging adjacent tissue such as puncturing the rectum R or the bladder B. Ultrasound probe connector 114 is used to secure the biopsy needle guide 100 to the ultrasound probe UP. The ultrasound probe connector 114 and the sliding bracket 102 may be axially moved along arm 112 to move the assembly closer to the perineum or away from the perineum as desired as indicated by the arrows. Locking element 116 secures the ultrasound probe connector 114 and sliding bracket into position relative to the perineum. Needle guide bracket 106 is adjustable up or down along slot 104 in sliding bracket 102 to move the needle guide bracket 106 either anteriorly (towards the scrotum) or posteriorly (towards the anus). The needle bracket may also be pivoted to control the angle of the biopsy needle as it enters the skin. Once in the desired position, the needle guide bracket 106 may be locked into position with locking element 110. A biopsy guide needle BN is then inserted into channel 108 (with or without optional sharp inner obturator, not illustrated), through the perineum avoiding the rectum and other anatomical areas into a desired area of the prostate using ultrasound guidance, and then the obturator is removed if used and a biopsy needle is inserted through the biopsy guide needle to biopsy the tissue and obtain a sample. The biopsy needle is removed, the sample collected and then the procedure may be repeated at a different location in the prostate by adjusting the position of the needle guide either through the initial puncture site or through a new puncture site. Additional tissue samples may be obtained by adjusting the biopsy guide needle position and then reinserting a biopsy needle, but without removing the biopsy guide needle from the perineum, thereby allowing multiple samples to be obtained from different locations without requiring multiple needle penetrations through the perineum. Thus, the biopsy needle position may be easily positioned and repositioned without requiring uncoupling of the biopsy needle from the biopsy guide device.

Upon completion of the biopsy procedure. The biopsy needle guide may be disassembled, cleaned and then re-sterilized for another procedure. Because the device may be reusable, the cost per procedure is less than other devices. Other examples of the device may be single use only. The device may be formed from metals such as stainless steel, titanium, cobalt-chromium alloy, etc. The device may be formed from polymers such as PVC, ABS, polystyrene, PEEK, or any other materials known in the art. The device may be formed from combinations of metals and polymers, or any other materials used in the surgical instrument arts.

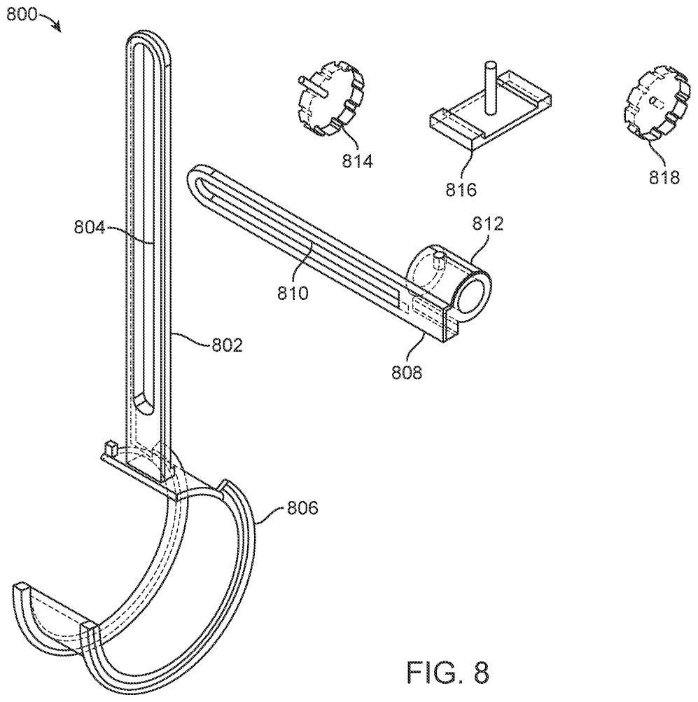

FIG. 8 illustrates another example of a biopsy guide 800 that is generally the same as the example in FIG. 1 with the major difference being the ultrasound probe connector element 806 is an open ring configured to accommodate various sizes of ultrasound probes. Other elements of the device include the sliding bracket 802, slot 804, arm 808 with slot 810, needle guide bracket 812, and locking mechanism 814, 816, 818 which secures the sliding bracket 802 and ultrasound probe connector 806 to the arm 808. Here, the needle bracket 812 is integrated with the arm 808 unlike the previous example where they are separate.

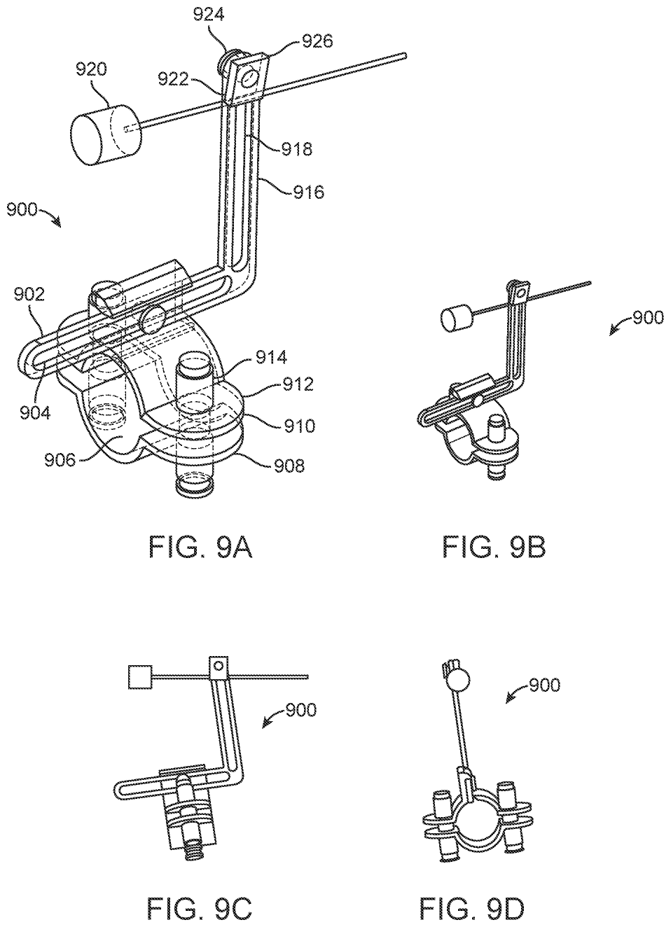

FIGS. 9A-9D illustrate another example of a biopsy guide 900 that is similar to the example in FIG. 1 with the major difference being that the ultrasound probe connector 910 includes a bracket having a concave upper half 912 and a concave lower half 908 with pins 914 that join the two halves together. This allows the clamp created by the two concave halves to form a receptacle that engages and captures a number of different ultrasound probe sizes and shapes in channel 906 formed by the opposed concave regions. Other aspects of the needle guide 900 are similar to that of FIG. 1.

In FIG. 9A, arm 902 includes longitudinal slot 904 so that the ultrasound connector can slide along the slot 904 and a set screw can lock the position when desired. Also, sliding bracket 916 is also integrally connected to arm 902 and has a slot 918 to allow needle bracket 926 to slide up and down the bracket 916. In this example, the slot 918 is orthogonal or transverse to the slot 904 in the arm 902 although any angle may be used. Set screw 924 locks the needle bracket in position along slot 918. Biopsy guide needle 920 can be inserted into channel 922 of the needle bracket 926 and a biopsy needle (not shown) can be inserted through the biopsy guide needle 920 when the desired position is established.

FIG. 9B shows another view of the guide 900 from FIG. 9A.

FIG. 9C illustrates pivoting of the needle bracket 926 relative to the sliding bracket 916 to adjust penetration angle of the guide needle and biopsy needle through the perineum. Set screw 924 may be loosened or tightened to permit or prevent rotation of the needle bracket 926 as well as its sliding along slot 918.

FIG. 9D illustrates rotation of the entire device relative to the longitudinal axis of the ultrasound probe to adjust the clock position of the needle guide bracket. Here, the needle guide bracket is rotated at a slight counter clockwise angle relative to the perpendicular line that extends from the ultrasound probe (not shown).

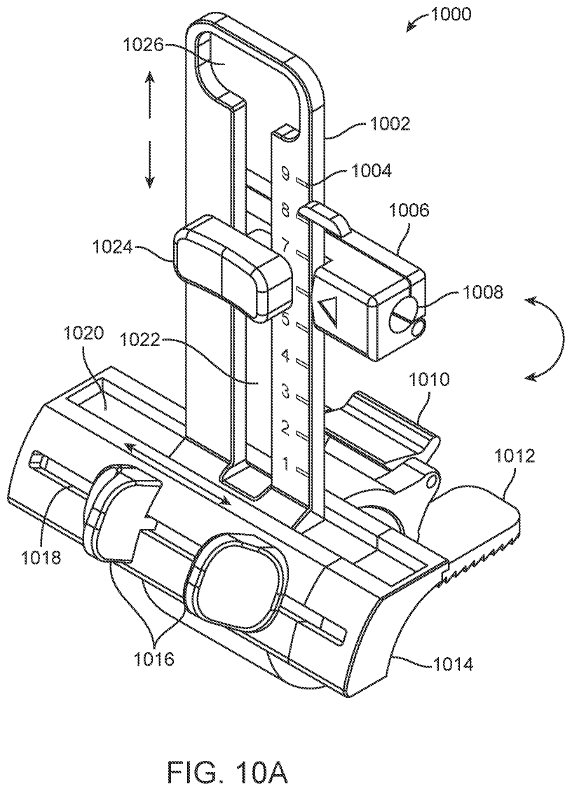

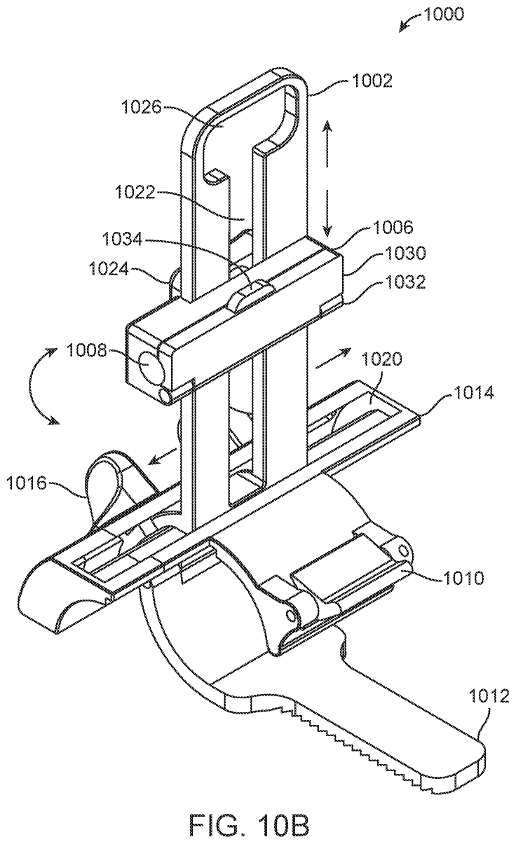

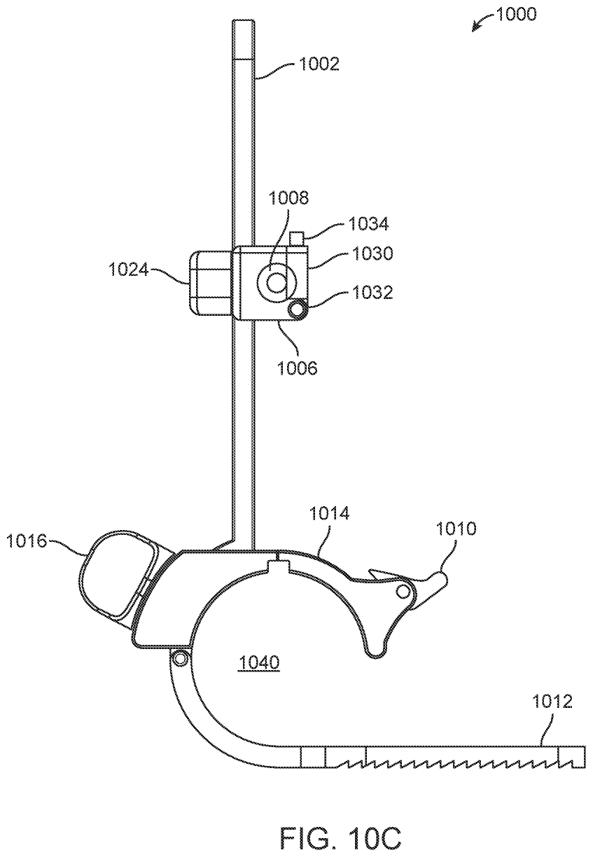

FIGS. 10A-10C illustrate another example of biopsy needle guide 1000 that is similar to previous examples with the major difference being that guide 1000 has different locking mechanisms for locking the position of the various components relative to one another or to permit their movement. Other differences will be evident in the disclosure below.

FIG. 10A shows a left perspective view of biopsy needle guide 1000 which includes bracket 1002 (sometimes also referred to as a slider or slider bracket) which is coupled to a base 1014. A needle guide bracket 1006 is coupled to bracket 1002, and locking mechanism 1024 allows the needle guide bracket 1006 to slide up and down bracket 1002 as well as pivot, or to fix the needle guide bracket 1006 in position relative to bracket 1002. Base 1014 supports bracket 1002 and also may be coupled to an ultrasound probe (not shown). Locking tabs 1016, here two, although any number may be used including one or more tabs, may be actuated to allow the bracket 1002 to slide along groove 1020 in base 1014 or to lock the bracket 1002 in position relative to base 1014. Adjustable and flexible band or strap 1012 may be disposed around an ultrasound probe (not shown) to secure base 1014 to the ultrasound probe and the free end of the adjustable band or strap 1012 may be fed through a pawl 1010 which can be opened or closed manually by an operator to allow the band to be tightened or loosened.

Bracket 1002 may include indicia 1004 which may be any symbol, color, tactile feature such as surface texturing, etc. to indicate the position of the needle guide bracket 1006 as it moves vertically up and down along slot 1022 as indicated by the vertical arrows. Slot 1022 includes an elongate rectangular slot that runs substantially parallel to the longitudinal axis of the bracket 1002, and also the upper end of the slot optionally includes an enlarged rectangular shaped head slotted region 1026. This enlarged head allows locking mechanism 1024 and needle guide bracket 1006 to be slid into the enlarged head region and then decoupled from the bracket 1002 which may be desirable for cleaning if the device 1000 is reusable.

Needle guide bracket 1006 includes elongate channel 1008 that extends through the bracket 1006 and is substantially parallel to the longitudinal axis of bracket 1006. A biopsy needle guide with or without a sharp inner obturator (not shown) may be inserted into channel 1008 for introduction into the target tissue such as a prostate, and then the obturator is removed if used and the biopsy needle may be inserted through the biopsy needle guide to obtain a sample of the target tissue such the prostate. Optionally in this or any example, the coaxial biopsy needle guide (not shown) may snap fit or friction fit into position in the central channel 1008. The coaxial biopsy needle guide may have a tapered outer surface to help create the friction fit and prevent it from sliding out. This may obviate the need for a rubber sleeve that is often placed over the biopsy needle guide to create the friction fit. The needle guide bracket 1006 may include an optional openable and closeable door to provide access to channel 1008 for cleaning purposes if desired, or the needle guide bracket 1006 may be a single integral piece.

Locking mechanism 1024 here is a rectangular button which may be depressed to release the needle guide bracket 1006 from bracket 1002 to allow the needle guide bracket to slide up and down along slot 1022 as indicated by the vertical arrows. Also, locking mechanism 1024 when released allows the needle guide bracket 1006 to pivot clockwise or counterclockwise to change the entry angle of the biopsy needle when introduced into the perineum, as indicated by the curved arrow. Optional indicia on the needle guide bracket such as an arrowhead indicate the direction of the device that faces toward the patient for orientation purposes. Therefore, as previously discussed in examples above, the channel 1008 may be parallel with the base and the ultrasound probe, or the channel 1008 may be angled upward or downward relative to the base and ultrasound probe. For example, the channel may be angled up to +90 degrees away from the base 1014, or angled downward toward the base up to −90 degrees. The angle of the channel 1008 also is the same as the angle of the biopsy needle guide and biopsy needle once inserted in channel 1008. Therefore, the biopsy needle guide and biopsy needle may be parallel to the longitudinal axis of the ultrasound probe, or angled upward and away from the ultrasound probe or angled downward toward the ultrasound probe if pivoted.

Base 1014 includes a concave inner surface that is shaped to conform to at least a portion of an ultrasound transducer (not shown). A groove 1020 in the base 1014 allows the bracket 1002 to slide parallel to the longitudinal axis of the base 1014 as indicated by the arrows adjacent slot 1020, when locking mechanism 1016 is released. Locking mechanism 1016 here may include tabs which may slide along slot 1018 to release or lock bracket 1002 to either allow bracket 1002 to slide along base 1014 or to hold base in a desired position on base 1014. Additional details about device 1000 are disclosed below. Because the base 1014 is coupled to the ultrasound probe, the base lies substantially parallel to the longitudinal axis of the ultrasound probe and bracket 1002 will also slide substantially parallel to the longitudinal axis of the ultrasound probe. Bracket 1002 extends vertically away from the base 1014 and ultrasound probe, and may be orthogonal to or transverse to the base or ultrasound probe longitudinal axis.

FIG. 10B shows a perspective view of the device 1000 in FIG. 10A but this time illustrated from the opposite side, here a right side relative to the view in FIG. 10A. This view illustrates an optional openable door 1030 that may be included on needle guide bracket 1006. Here, the door 1030 is hingedly coupled to the needle guide bracket 1006 to allow the door to open and close exposing channel 1008. The hinge 1032 may include a pin extending through the needle guide bracket and the door to pivotally couple the two together. An optional tab 1034 on the door 1030 allows an operator to easily grasp the door so it can be opened and closed. The door may have a snap fit, magnetic lock, or other locking mechanism that keeps the door closed and engaged with the needle guide bracket 1006 when closed. Opening the door allows the channel 1008 to be cleaned if needed such as in the case when the device is reusable, or if access to the channel or biopsy needle guide is desired. In examples where the device is a single use device, the door may not be needed since the device is used once and then discarded, or access may still be desired therefore the optional door may be included with single use as well as reusable examples of the device.

FIG. 10C shows a front end view of the device 1000 illustrated in FIGS. 10A-10B. The annular region 1040 is more clearly visible in this view. An ultrasound probe (not shown) may be inserted into annular region 1040 and then the probe is secured to the device 1000 by inserting strap 1012 into the pawl 1010 where it may be tightened. Teeth in strap 1012 engage the pawl to prevent loosening to maintain the strap tightly around the ultrasound probe. When loosening of the strap 1012 is desired, pawl 1010 may be disengaged from the teeth by pivoting the pawl to disengage the teeth so as to allow the strap to be pulled out of pawl 1010.

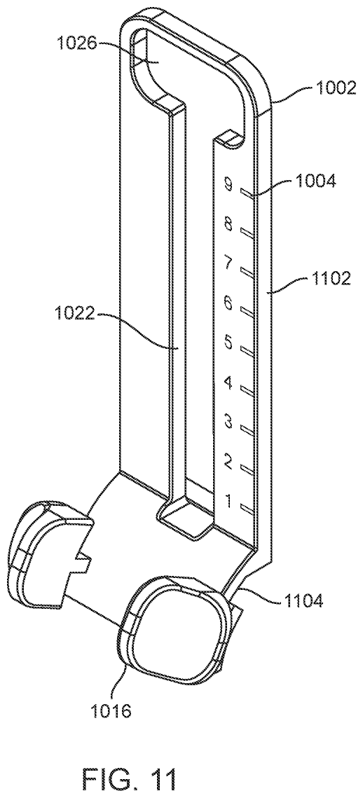

FIG. 11 shows a perspective view of the slider or bracket 1002 uncoupled from the rest of the device 1000. The bracket 1002 includes an elongate flat planar vertical section 1102 which extends away from an angled section 1104 to which the locking mechanism 1016 is coupled. The angled section is shaped to conform to the base 1014 so that the bracket 1002 can slide smoothly along the longitudinal axis of the base 1014 while the vertical section 1102 slides along groove 1020 in base 1014 (best seen in FIGS. 10A-10C). Other aspects of bracket 1002 have previously been described above.

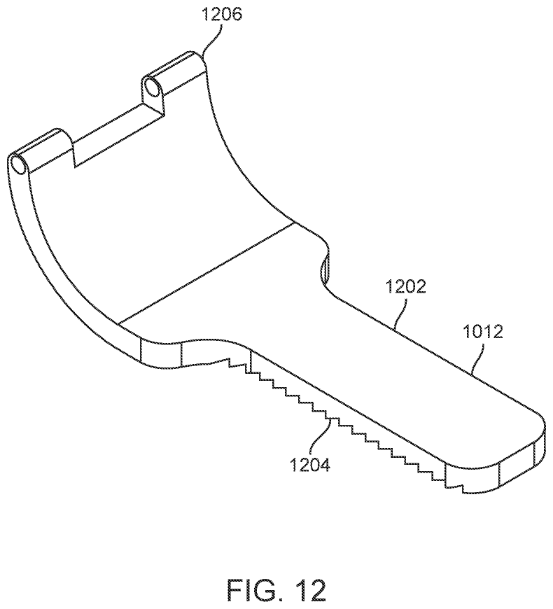

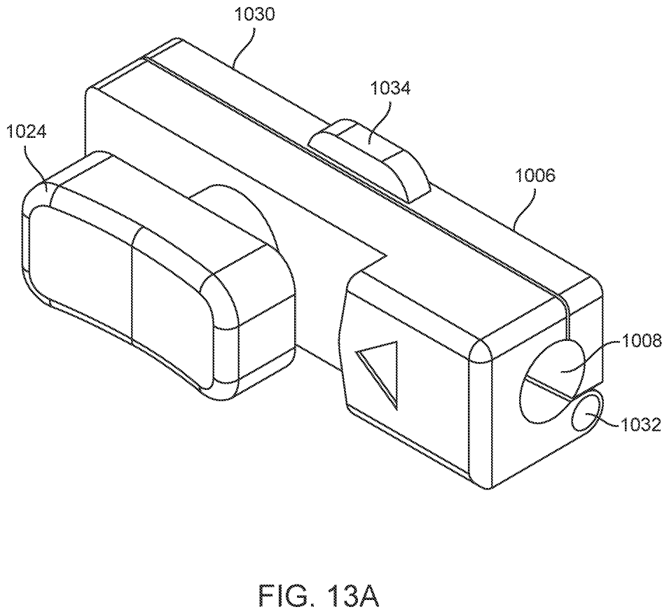

FIG. 12 shows the band (also referred to herein as flexible band or strap) 1012 which is coupled to the base 1014 at one end and the pawl 1010 at the opposite end to form adjustable annular region 1040 which holds the ultrasound probe. The band 1012 may include a resilient tongue section 1202 with teeth 1204 on an outer surface that allows locking when the tongue is inserted into the pawl on the base. Thus, the tongue may be disposed around an ultrasound probe and tightened to couple the base 1014 and entire device 1000 to the ultrasound probe. The inner surface of the tongue may be smooth or textured to ensure better gripping and engagement with the ultrasound probe. One end of the band 1012 may include a hinged region 1206 which includes a castellated region with a receiving region between adjacent protruding shoulder members. The protruding shoulder members include a hole extending through them in order to receive a pin, and the receiving region receives a protruding member from the base 1014. The pin (not illustrated) couples the hinged end of the band with the base 1014 and allows pivoting therebetween similar to a wristwatch band. Other hinge mechanisms may be used, or in some examples the material itself is sufficiently resilient so that it serves as the hinge and a separate hinge mechanism is not required.

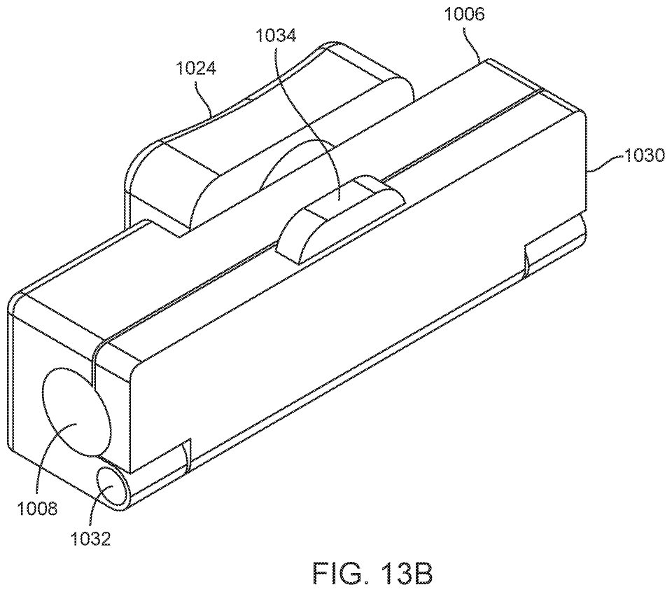

FIGS. 13A-13C show the needle guide bracket 1006 of device 1000.

FIG. 13A is left perspective view of the needle guide bracket 1006 previously described in FIGS. 10A-10C. The needle guide bracket 1006 is a generally rectangular housing with a central channel 1008 extending though the housing that is sized to receive a biopsy needle guide. Other shapes for the housing may be utilized and therefore a rectangular shape is not intended to be limiting. Lock 1024 is a rectangular shaped button which can be actuated to release the needle guide bracket 1006 and allow it to slide along slot 1022 on bracket 1002. The lock 1024 in its unbiased position holds the needle guide bracket 1006 in a desired position. The needle guide bracket may be moved either in discrete steps along the slot 1022, or it may be moved continuously along slot 1022 into any desired position. Additionally, actuation of lock button 1024 allows the needle guide bracket to pivot clockwise or counterclockwise so as to change the angle of the longitudinal axis of the needle guide bracket relative to the longitudinal axis of the base 1014. As mentioned above, this angle may be roughly as high as +90 degrees up or as low as −90 degrees down. Channel 1008 is sized to snugly receive a biopsy needle guide and hold it in position. Optionally, an openable and closeable door 1030 is coupled to the housing with hinge 1032, here a pin through apertures in the door and the housing may be included. The door allows access to channel 1008 for cleaning or any other reason if desired, especially in the example where the device is reusable. If the device is a single use device the access to channel 1008 may not be needed and therefore the door may not be included and the housing is a single integral piece or several components coupled together. Tab 1034 allows an operator to easily grasp and open or close the door, which may be secured in position with a snap fit to the housing. An optional arrow or other indicia on the surface of the housing may provide a visual or tactile indicator of the direction in which the needle should be advanced into the target tissue.

FIG. 13B is a right perspective view of the needle guide bracket 1006 illustrated in FIG. 13A. It highlights the optional door 1030 and hinge 1032 which may be a pin in an aperture in protrusions from the housing and the door, similar to a standard door hinge.

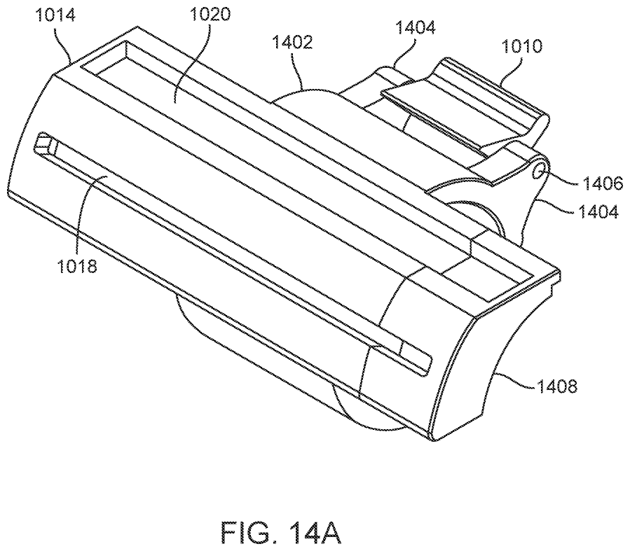

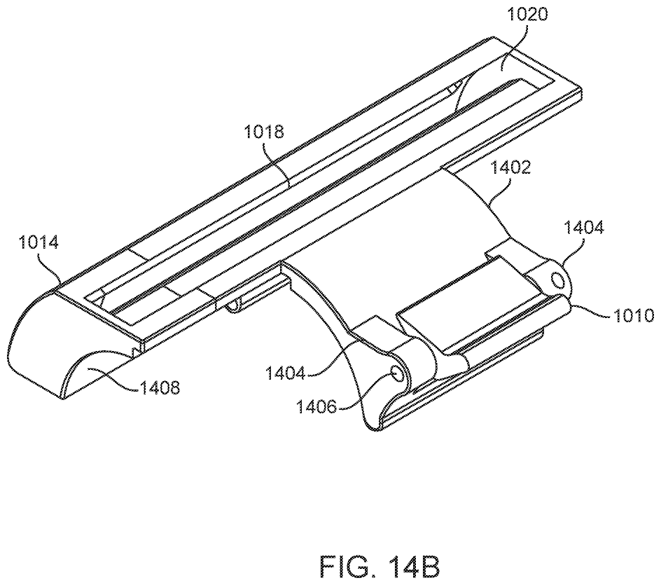

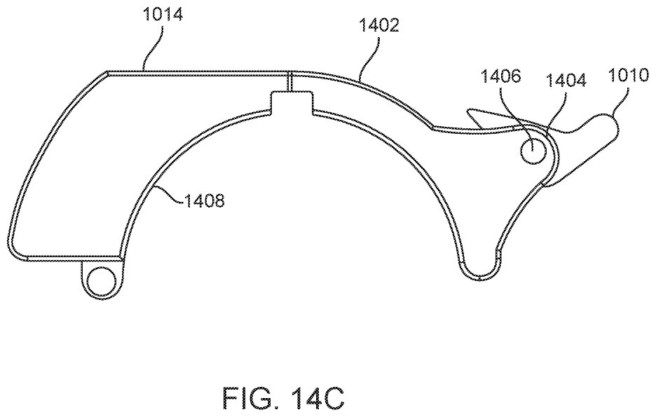

FIGS. 14A-14C illustrate the base 1014 previously described in the device 1000 shown in FIGS. 10A-10C above. FIG. 14A shows a left perspective of the base 1014 which includes an elongate rectangular slot 1020 that runs parallel with the longitudinal axis of the base 1014 and therefore is also substantially parallel with the longitudinal axis of the ultrasound probe to which the base is coupled. The slot 1020 allows bracket 1002 to slide along the slot thereby moving the biopsy needle bracket closer to or away from the perineum of the patient. Another elongate slot 1018 that is parallel with the elongate slot 1020 and therefore parallel with the longitudinal axis of the base 1014 and the ultrasound probe (not shown) allows the tabs 1016 of the lock mechanism to slide along the slot to permit movement of bracket 1002. The tabs may be in an unbiased position where they are locked to prevent movement of the bracket along the base.

An inner surface 1408 of the base may be concave to help the inner surface conform to the ultrasound probe to which it is coupled. Additionally, a portion 1402 of the base may extend outward from the right side and have an inner concave surface also to help ensure the base conforms to the ultrasound probe and also to provide a coupling point for the strap. A pivotable pawl 1010 allows the strap to be adjustably coupled to the base. Here, the pawl 1010 is hingedly coupled to two protruding shoulders 1404 with a pin 1406 that passes through a channel in the pawl and the shoulders. Thus, when the pawl is deflected by the operator, the strap may be inserted into the gap between the pawl and the base and advanced to tighten the strap against the ultrasound probe. And in the unbiased position the pawl will rest against teeth on the strap to prevent the strap from moving and loosening.

FIG. 14B shows a right perspective view of the base 1014 in FIG. 14A.

FIG. 14C is a front-end view of the base 1014.

The device illustrated in FIGS. 10A-14C may be used in a similar manner as previously described and illustrated in FIG. 7 above with the major difference being that instead of set screws used to release or lock the various components, the example in FIGS. 10A-14C use difference locking mechanisms. Movement of the device along its axes is generally the same for all examples disclosed herein. That movement may include movement of the needle guide bracket inward toward and outward away from the perineum as well as pivoting of the needle guide bracket upward and downward relative to the axis of the ultrasound probe, and also up and down motion of the needle guide toward the scrotum or toward the perineum.

As previously disclosed, in any example, the needle guide bracket may be used to hold a guide needle. In some cases, a sharp inner cannula is inserted into the guide needle to help pierce tissue and then the inner cannula is removed, and a biopsy needle is inserted into the guide needle to obtain a tissue sample. In any example, the sharp inner cannula may not be needed and the guide needle is sharp enough and strong enough to pierce the tissue and then the biopsy needle may be inserted into the guide needle to obtain the tissue sample without using the inner cannula. Also, in any example, the coaxial biopsy needle guide may snap fit or friction fit into position in the central channel of the needle guide bracket. The coaxial biopsy needle guide may have a tapered outer surface to help create the friction fit and prevent it from sliding out. This may obviate the need for a rubber sleeve that is often placed over the biopsy needle guide to create the friction fit.

### Notes and Examples

The following, non-limiting examples, detail certain aspects of the present subject matter to solve the challenges and provide the benefits discussed herein, among others.

Example 1 is a biopsy needle guide comprising an ultrasound probe coupling element configured to be coupled to an ultrasound probe; a sliding bracket coupled with the ultrasound probe coupling element; an elongate arm, wherein the ultrasound probe coupling element and the sliding bracket are operably coupled to the arm, and wherein the ultrasound probe coupling element and the sliding bracket are slidably movable along the elongate arm; and a needle guide bracket slidably movable along the sliding bracket, the needle guide bracket configured to hold a biopsy needle or a biopsy needle guide.

Example 2 is the biopsy needle guide of Example 1, further comprising the biopsy needle.

Example 3 is the biopsy needle guide of any of Examples 1-2, further comprising a biopsy guide needle disposed in a channel of the needle guide bracket and wherein the biopsy needle is slidably and coaxially disposed in the biopsy guide needle.

Example 4 is the biopsy needle guide of any of Examples 1-3, wherein the ultrasound probe coupling element comprises an adjustable ring.

Example 5 is the biopsy needle guide of any of Examples 1-4, wherein the sliding bracket is fixedly coupled with the ultrasound probe coupling element.

Example 6 is the biopsy needle guide of any of Examples 1-5, wherein slidable movement of the sliding bracket along the elongate arm is configured to move the needle guide bracket closer to or further away from a perineum of a patient.

Example 7 is the biopsy needle guide of any of Examples 1-6, wherein slidable movement of the needle guide bracket along the sliding bracket is configured to move the needle guide bracket closer to an anus of a patient and away from a perineum of the patient, or away from the anus and closer to the perineum.

Example 8 is the biopsy needle guide of any of Examples 1-7, wherein the sliding bracket comprises an elongate slot disposed axially along a longitudinal axis of the sliding bracket, and wherein the needle guide bracket slides along the elongate slot.

Example 9 is the biopsy needle guide of any of Examples 1-8, wherein the needle guide bracket is pivotably coupled to the sliding bracket.

Example 10 is the biopsy needle guide of any of Examples 1-9, further comprising a releasable locking element coupled to the needle guide bracket and the sliding bracket, the releasable locking element is configured to hold the needle guide bracket in a fixed position relative to the sliding bracket.

Example 11 is the biopsy needle guide of any of Examples 1-10, further comprising a releasable locking element coupled to the arm and the ultrasound probe coupling element or the sliding bracket, the releasable locking element configured to hold the ultrasound probe coupling element and the sliding bracket in a fixed position relative to the arm.

Example 12 is the biopsy needle guide of any of Examples 1-11, wherein the needle guide comprises a channel for holding the biopsy needle or the biopsy needle guide, and wherein the channel is parallel to a longitudinal axis of the ultrasound probe.

Example 13 is the biopsy needle guide of any of Examples 1-12, wherein the biopsy needle is adjustable without requiring removal of the biopsy needle from the needle guide bracket.

Example 14 is a system for biopsying tissue comprising the biopsy needle guide of any of Examples 1-13 and the ultrasound probe.

Example 15 is a method for biopsying tissue comprising providing a biopsy needle guide comprising an ultrasound probe coupling element, a sliding bracket, an elongate arm, a needle guide bracket, and a biopsy guide needle; releasably coupling the ultrasound probe coupling element with an ultrasound probe; inserting the ultrasound probe into an anus of a patient; moving the coupling element and the sliding bracket toward a perineum of the patient or away from the perineum by sliding the coupling element and the sliding bracket along the elongate arm; moving the needle guide closer to or away from the anus and correspondingly moving the needle guide away from or closer to the perineum by sliding the needle guide along the sliding bracket; pivotably adjusting the needle guide by rotating the needle guide relative to the sliding bracket; fixing the needle guide relative to the sliding bracket and fixing the ultrasound probe coupling element and the sliding bracket relative to the elongate arm; inserting a biopsy guide needle through the needle guide bracket and through the perineum into a prostate; and inserting a biopsy needle through the biopsy guide needle to obtain a tissue sample.

Example 16 is the method of Example 15, wherein releasably coupling the ultrasound probe coupling element with the ultrasound probe comprises inserting the ultrasound probe into an adjustable ring.

Example 17 is the method of any of Examples 15-16, further comprising imaging the biopsy needle during the insertion with the ultrasound probe.

Example 18 is the method of any of Examples 15-17, wherein moving the needle guide comprises slidably moving the needle guide along a slot disposed in the sliding bracket.

Example 19 is the method of any of Examples 15-18, wherein fixing the needle guide comprises adjusting a set screw.

Example 20 is a biopsy needle guide comprising a base having a concave inner surface configured to conform with an ultrasound probe and a slotted region extending along a longitudinal axis of the base, the base further comprising a first locking mechanism; a flexible strap having a first end coupled to the base and a second free end coupled to the base with the first locking mechanism; a bracket slidably disposed in the slotted region in the base, the bracket comprising a slotted region; a needle guide bracket slidably disposed in the slotted region of the bracket, and wherein the needle guide bracket comprises a central channel extending along a longitudinal axis of the needle guide bracket, the central channel configured to receive a biopsy needle guide; a second locking mechanism coupled to the base, wherein actuation of the second locking mechanism permits the bracket to slide along the slotted region in the base; and a third locking mechanism coupled to the needle guide bracket, wherein actuation of the third locking mechanism permits the needle guide bracket to slide in the slotted region of the bracket and permits the needle guide bracket to pivot.

Example 21 is the biopsy needle guide of Example 20, wherein the slotted region in the base is an elongate rectangular slotted region.

Example 22 is the biopsy needle guide of any of Examples 20-21, wherein sliding the bracket along the slotted region in the base moves the needle guide bracket closer to or further away from a patient's perineum.

Example 23 is the biopsy needle guide of any of Examples 20-22, wherein the second locking mechanism coupled to the base comprises a pair of opposable tabs.

Example 24 is the biopsy needle guide of any of Examples 20-23, wherein the pair of opposable tabs slide along a slotted region in the base that extends parallel to the longitudinal axis of the base.

Example 25 is the biopsy needle guide of any of Examples 20-24, wherein the first locking mechanism on the base comprises a pawl configured to receive the flexible strap.

Example 26 is the biopsy needle guide of any of Examples 20-25, wherein the flexible strap comprises a plurality of teeth configured to engage and lock with the first locking mechanism in the base.

Example 27 is the biopsy needle guide of any of Examples 20-26, wherein the slotted region in the bracket is orthogonal to the longitudinal axis of the base.

Example 28 is the biopsy needle guide of any of Examples 20-27, wherein sliding the needle guide bracket along the bracket is configured to move the needle guide bracket closer to a patient's scrotum and away from the patient's anus, or away from the patient's scrotum and closer to the patient's anus.

Example 29 is the biopsy needle guide of any of Examples 20-28, wherein the bracket comprises indicia for indicating a position of the needle guide bracket on the bracket.

Example 30 is the biopsy needle guide of any of Examples 20-29, wherein the slotted region in the bracket comprises an elongated rectangular slotted region coupled to an enlarged head slotted region.

Example 31 is the biopsy needle guide of any of Examples 20-30, wherein the needle guide bracket comprises an openable and closeable door that provides access to the central channel.

Example 32 is the biopsy needle guide of any of Examples 20-31, wherein the third locking mechanism comprises an actuatable button.

Example 33 is a system for performing a biopsy, comprising the biopsy needle guide of any of Examples 20-32 and the ultrasound probe.

Example 34 is the system of Example 33, further comprising a biopsy guide needle, a sharp obturator disposed in the biopsy needle guide, or a biopsy needle slidably disposed in the central channel of the needle guide bracket.

Example 35 is a method of performing a biopsy on tissue, the method comprising providing a biopsy needle guide comprising a base, a bracket coupled to the base, a flexible strap coupled to the base, and a needle guide bracket coupled to the bracket; releasably coupling an ultrasound probe to the base with the flexible strap; inserting the ultrasound probe into an anus of a patient; slidably moving the bracket closer to or further away from a perineum of the patient; slidably adjusting the needle guide bracket to a position closer to or further away from the anus and correspondingly away from or closer to the perineum; pivoting the needle guide bracket relative to the bracket; locking the bracket and the needle guide bracket in position; inserting a biopsy guide needle into a channel in the needle guide bracket and through the perineum into a prostate of the patient; and inserting a biopsy needle through the biopsy guide needle to obtain a tissue sample.

Example 36 is the method of Example 35, further comprising imaging the biopsy needle during the insertion with the ultrasound probe.

Example 37 is the method of any Examples 35-36, wherein releasably coupling the ultrasound probe comprises engaging a pawl on the base with one or more teeth on the flexible strap.

Example 38 is the method of any of Examples 35-37, wherein slidably moving the bracket comprises actuating a locking mechanism to unlock the bracket and sliding the bracket along a slot disposed in the base.

Example 39 is the method of any of Examples 35-38, wherein slidably moving the needle guide bracket comprises actuating a locking mechanism to unlock the needle guide bracket and sliding the needle guide bracket along a slot disposed in the bracket.

Example 40 is the method of any of Examples 35-39, wherein pivoting the needle guide bracket comprises actuating a locking mechanism to unlock the needle guide bracket and pivoting the needle guide bracket relative to the bracket.

Example 41 is the method of any of Examples 35-40, further comprising opening a door on the needle guide bracket to provide access to the channel.

In Example 42, the devices or methods of any one or any combination of Examples 1-41 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A biopsy needle guide comprising:
an ultrasound probe coupling element configured to be coupled to an ultrasound probe;
a sliding bracket coupled with the ultrasound probe coupling element;
an elongate arm, wherein the ultrasound probe coupling element and the sliding bracket are operably coupled to the arm, and wherein the ultrasound probe coupling element and the sliding bracket are slidably movable along the elongate arm; and
a needle guide bracket slidably movable along the sliding bracket, the needle guide bracket configured to hold a biopsy needle or a biopsy needle guide.

2. The biopsy needle guide of claim 1, further comprising the biopsy needle.

3. The biopsy needle guide of claim 2, further comprising a biopsy guide needle disposed in a channel of the needle guide bracket and wherein the biopsy needle is slidably and coaxially disposed in the biopsy guide needle.

4. The biopsy needle guide of claim 1, wherein the ultrasound probe coupling element comprises an adjustable ring.

5. The biopsy needle guide of claim 1, wherein the sliding bracket is fixedly coupled with the ultrasound probe coupling element.

6. The biopsy needle guide of claim 1, wherein slidable movement of the sliding bracket along the elongate arm is configured to move the needle guide bracket closer to or further away from a perineum of a patient.

7. The biopsy needle guide of claim 1, wherein slidable movement of the needle guide bracket along the sliding bracket is configured to move the needle guide bracket closer to an anus of a patient and away from a perineum of the patient, or away from the anus and closer to the perineum.

8. The biopsy needle guide of claim 1, wherein the sliding bracket comprises an elongate slot disposed axially along a longitudinal axis of the sliding bracket, and wherein the needle guide bracket slides along the elongate slot.

9. The biopsy needle guide of claim 1, wherein the needle guide bracket is pivotably coupled to the sliding bracket.

10. The biopsy needle guide of claim 1, further comprising a releasable locking element coupled to the needle guide bracket and the sliding bracket is configured to hold the needle guide bracket in a fixed position relative to the sliding bracket.

11. The biopsy needle guide of claim 1, further comprising a releasable locking element coupled to the arm and the ultrasound probe coupling element or the sliding bracket, the releasable locking element configured to hold the ultrasound probe coupling element and the sliding bracket in a fixed position relative to the arm.

12. The biopsy needle guide of claim 1, wherein the needle guide comprises a channel for holding the biopsy needle or the biopsy needle guide, and wherein the channel is parallel to a longitudinal axis of the ultrasound probe.

13. The biopsy needle guide of claim 1, wherein the biopsy needle is adjustable without requiring removal of the biopsy needle from the needle guide bracket.

14. A system for biopsying tissue, comprising:
the biopsy needle guide of claim 1; and
the ultrasound probe.

15. A method for biopsying tissue, comprising:
providing a biopsy needle guide comprising an ultrasound probe coupling element, a sliding bracket, an elongate arm, a needle guide bracket, and a biopsy guide needle;
releasably coupling the ultrasound probe coupling element with an ultrasound probe;
inserting the ultrasound probe into an anus of a patient;
moving the coupling element and the sliding bracket toward a perineum of the patient or away from the perineum by sliding the coupling element and the sliding bracket along the elongate arm;
moving the needle guide bracket closer to or away from the anus and correspondingly moving the needle guide bracket away from or closer to the perineum by sliding the needle guide bracket along the sliding bracket;
pivotably adjusting the needle guide bracket by rotating the needle guide bracket relative to the sliding bracket;
fixing the needle guide bracket relative to the sliding bracket and fixing the ultrasound probe coupling element and the sliding bracket relative to the elongate arm; and
inserting the biopsy guide needle through the needle guide bracket and through the perineum into a prostate;
inserting a biopsy needle through the biopsy guide needle to obtain a tissue sample.

16. The method of claim 15, wherein releasably coupling the ultrasound probe coupling element with the ultrasound probe comprises inserting the ultrasound probe into an adjustable ring.

17. The method of claim 15, further comprising imaging the biopsy needle during the insertion with the ultrasound probe.

18. The method of claim 15, wherein the moving the needle guide bracket comprises slidably moving the needle guide along a slot disposed in the sliding bracket.

19. The method of claim 15, wherein the fixing the needle guide bracket or the biopsy needle comprises adjusting a set screw.

20. A biopsy needle guide, comprising:
a base having a concave inner surface configured to conform with an ultrasound probe and a slotted region extending along a longitudinal axis of the base, the base further comprising a first locking mechanism;
a flexible strap having a first end coupled to the base and a second free end coupled to the base with the first locking mechanism;
a bracket slidably disposed in the slotted region in the base, the bracket comprising a slotted region;
a needle guide bracket slidably disposed in the slotted region of the bracket, and wherein the needle guide bracket comprises a central channel extending along a longitudinal axis of the needle guide bracket, the central channel configured to receive a biopsy needle guide;
a second locking mechanism coupled to the base, wherein actuation of the second locking mechanism permits the bracket to slide along the slotted region in the base; and
a third locking mechanism coupled to the needle guide bracket, wherein actuation of the third locking mechanism permits the needle guide bracket to slide in the slotted region of the bracket and permits the needle guide bracket to pivot.

21. The biopsy needle guide of claim 20, wherein the slotted region in the base is an elongate rectangular slotted region.

22. The biopsy needle guide of claim 20, wherein sliding the bracket along the slotted region in the base moves the needle guide bracket closer to or further away from a patient's perineum.

23. The biopsy needle guide of claim 20, wherein the second locking mechanism coupled to the base comprises a pair of opposable tabs.

24. The biopsy needle guide of claim 23, wherein the pair of opposable tabs slide along a slotted region in the base that extends parallel to the longitudinal axis of the base.

25. The biopsy needle guide of claim 20, wherein the first locking mechanism on the base comprises a pawl configured to receive the flexible strap.

26. The biopsy needle guide of claim 20, wherein the flexible strap comprises a plurality of teeth configured to engage and lock with the first locking mechanism in the base.

27. The biopsy needle guide of claim 20, wherein the slotted region in the bracket is orthogonal to the longitudinal axis of the base.

28. The biopsy needle guide of claim 20, wherein sliding the needle guide bracket along the bracket is configured to move the needle guide bracket closer to a patient's scrotum and away from the patient's anus, or away from the patient's scrotum and closer to the patient's anus.

29. The biopsy needle guide of claim 20, wherein the bracket comprises indicia for indicating a position of the needle guide bracket on the bracket.

30. The biopsy needle guide of claim 20, wherein the slotted region in the bracket comprises an elongated rectangular slotted region coupled to an enlarged head slotted region.

31. The biopsy needle guide of claim 20, wherein the needle guide bracket comprises an openable and closeable door that provides access to the central channel.

32. The biopsy needle guide of claim 20, wherein the third locking mechanism comprises an actuatable button.

33. A system for performing a biopsy, comprising:
the biopsy needle guide of claim 20; and
the ultrasound probe.

34. The system of claim 33, further comprising a biopsy guide needle, a sharp obturator disposed in the biopsy needle guide, or a biopsy needle slidably disposed in the central channel of the needle guide bracket.

35. A method of performing a biopsy on tissue, the method comprising:
providing a biopsy needle guide comprising a base, a bracket coupled to the base, a flexible strap coupled to the base, and a needle guide bracket coupled to the bracket;
releasably coupling an ultrasound probe to the base with the flexible strap;
inserting the ultrasound probe into an anus of a patient;
slidably moving the bracket closer to or further away from a perineum of the patient;
slidably adjusting the needle guide bracket to a position closer to or further away from the anus and correspondingly away from or closer to the perineum;
pivoting the needle guide bracket relative to the bracket;
locking the bracket and the needle guide bracket in position;
inserting a biopsy guide needle into a channel in the needle guide bracket and through the perineum into a prostate of the patient; and
inserting a biopsy needle through the biopsy guide needle to obtain a tissue sample.

36. The method of claim 35, further comprising imaging the biopsy needle during the insertion with the ultrasound probe.

37. The method of claim 35, wherein releasably coupling the ultrasound probe comprises engaging a pawl on the base with one or more teeth on the flexible strap.

38. The method of claim 35, wherein slidably moving the bracket comprises actuating a locking mechanism to unlock the bracket and sliding the bracket along a slot disposed in the base.

39. The method of claim 35, wherein slidably moving the needle guide bracket comprises actuating a locking mechanism to unlock the needle guide bracket and sliding the needle guide bracket along a slot disposed in the bracket.

40. The method of claim 35, wherein pivoting the needle guide bracket comprises actuating a locking mechanism to unlock the needle guide bracket and pivoting the needle guide bracket relative to the bracket.

41. The method of claim 35, further comprising opening a door on the needle guide bracket to provide access to the channel.
